(19) United States
(12) Patent Application Publication
(10) Pub. No.: US 2009/0253706 A1
(43) Pub. Date: Oct. 8, 2009

(54) Methods and Compositions for the Treatment of Rheumatoid Arthritis and Other Inflammatory Diseases

(76) Inventors: Alexander J. Muller, Media, PA (US); Laura Mandik-Nayak, Plymouth Meeting, PA (US); Lisa Laury-Kleintop, Ambler, PA (US); Goerge C. Prendergast, Penn Valley, PA (US); James B. DuHadaway, Wilmington, DE (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/418,173

(22) Filed: Apr. 3, 2009

Related U.S. Application Data
(60) Provisional application No. 61/042,089, filed on Apr. 3, 2008.

(51) Int. Cl. A61K 31/4985; A61P 19/02; A61P 29/00

(52) U.S. Cl. 514/249

(57) ABSTRACT

Compositions and methods for the treatment of inflammatory diseases are disclosed.

This application claims priority under 35 U.S.C. §119(e) to U.S. Provisional Patent Application No. 61/042,089, filed on Apr. 3, 2008. The foregoing application is incorporated by reference herein.

### FIELD OF THE INVENTION

This invention relates to the field of inflammatory diseases. Specifically, the invention provides novel compositions and methods for the treatment of inflammatory diseases.

### BACKGROUND OF THE INVENTION

Indoleamine 2,3-dioxygenase encoded by the Indo gene and herein referred to as IDO1 is an extrahepatic oxidoreductase that catalyzes the initial and rate-limiting step in the degradation of tryptophan along the kynurenine pathway that leads to the biosynthesis of nicotinamide adenine dinucleotide (NAD⁺) (Sono et al. (1996) Chem. Rev., 96:2841-87; Botting et al. (1995) Chem. Soc. Rev., 24:401-12; Sono et al. (1980) Biochem. Rev., 50:173-81). IDO1 is an IFN-γ target gene that has been suggested to play a role in immunomodulation (Mellor and Munn (1999) Immunol. Today, 20:469-473). Elevation of IDO1 activity depletes the levels of tryptophan in local cellular environments. Induction of IDO1 in antigen-presenting cells, where IDO1 is regulated by IFN-γ, blocks the activation of T cells, which are especially sensitive to tryptophan depletion. Recently, it has been shown that cytotoxic T cells become tolerized by a reduction in local concentrations of tryptophan that are elicited by IDO1 activity. Significantly, IDO1 activity has been shown to be elevated frequently in human tumors and/or in cancer patients (Yasui et al. (1986) Proc. Natl. Acad. Sci. USA. 83:6622-26; Taylor and Feng (1991) FASEB J. 5:2516-22). Since IDO1 can modulate immune responses, one implication is that IDO1 elevation in cancer may promote tumor immunosuppression (Mellor and Munn (1999) Immunol. Today, 20:469-473; Munn et al. (1999) J. Exp. Med. 189:1363-72; Munn et al. (1998) Science. 281:1191-93). A resultant hypothesis from this data was that if IDO1 drives cancer progression by blunting T cell activation, then IDO1 inhibition in animals should blunt tumor growth by reversing IDO1-mediated immunosuppression.

Recently, a second indoleamine 2,3-dioxygenase-related enzyme encoded by the Indol1 gene and referred to herein as IDO2, has been characterized and implicated as playing a role in the same pathways as IDO1 (see, e.g., PCT/US07/69271).

### SUMMARY OF THE INVENTION

In accordance with one aspect of the instant invention, methods for treating and/or inhibiting the onset of an inflammatory disease in patients in need thereof are provided. The methods comprise the administration of at least one inhibitor of tryptophan degradation. In a particular embodiment, the methods comprise the administration of a composition comprising at least one inhibitor of IDO1 and/or IDO2 and at least one pharmaceutically acceptable carrier. In a particular embodiment, the methods further comprise the administration of at least one additional anti-inflammatory agent concurrently and/or sequentially with the at least one inhibitor of IDO1 and/or IDO2. The methods may further comprise the administration of an immunosuppressive agent concurrently and/or sequentially with the at least one inhibitor of IDO1 and/or IDO2 and/or at least one additional anti-inflammatory agent.

Compositions for the treatment of inflammatory disease are also provided. The compositions comprise at least one inhibitor of IDO1 and/or IDO2 and at least one pharmaceutically acceptable carrier. In another embodiment, the composition further comprises at least one other anti-inflammatory compound and/or at least one immunosuppressive agent.

### BRIEF DESCRIPTION OF THE DRAWINGS

FIG. 1 is a graph of the mean ankle thickness over time of K/BxN mice which were treated with 1-methyl-D-tryptophan, 1-methyl-L-tryptophan, or carrier alone.

FIG. 2A is a graph of the titer of serum anti-glucose-6-phosphate isomerase (GPI) IgM and IgG isotypes from K/BxN mice treated with 1-methyl-tryptophan or carrier alone. FIG. 2B is a graph of the number of anti-GPI secreting cells per 10⁵ cells present in the spleen, draining lymph nodes (dLN), and non-draining lymph nodes (non-dLN) of K/BxN mice treated with 1-methyl-tryptophan or carrier alone.

FIG. 3 is a graph demonstrating that 1-methyl-D-tryptophan (D-1MT) inhibits arthritis development. K/BxN mice were treated orally (p.o.) with 400 mg/kg D-1MT, 400 mg/kg 1-methyl-D/L-tryptophan (D/L-MT) twice a day (b.i.d.), or carrier alone starting at 21 days. Rear ankles were measured as an indication of arthritis and represented as the mean ankle thickness±standard error (SEM). n=5 mice for each treatment.

FIG. 4 is a graph demonstrating the dose-dependent inhibition of arthritis development by methotrexate (MTX). K/BxN mice were treated weekly with MTX (1, 10, or 25 mg/kg) or carrier alone p.o. starting at the age of 3 weeks and followed for arthritis development. n=5 mice for each treatment group.

FIGS. 5A and 5B demonstrate that 1MT and MTX synergize to inhibit arthritis. K/BxN mice were treated with D/L-1MT (400 mg/kg p.o., b.i.d.), MTX (10 mg/kg (FIG. 5A) or 1 mg/kg (FIG. 5B) intraperitoneally (i.p.), 1×/wk), D/L-1MT+MTX, or carrier alone starting at the age of 3 weeks and followed for arthritis development. Rear ankles were measured as an indication of arthritis and represented as the mean ankle thickness±SEM. This is a representative experiment of 2 showing 5 mice for each treatment.

FIGS. 6A and 6B demonstrate that 1MT and MTX synergize to treat established arthritis. K/BxN mice were treated with D/L-1MT (400 mg/kg p.o., b.i.d.), MTX (10 mg/kg (FIG. 6A) or 1 mg/kg (FIG. 6B) i.p., weekly), D/L-1MT+MTX, or carrier alone starting after the onset of arthritis. Rear ankles were measured as an indication of arthritis and represented as the mean ankle thickness±SEM. n=5 mice for each treatment.

FIG. 7 provides a graph demonstrating attenuated arthritis in IDO1 deficient mice compared to wild-type mice.

### DETAILED DESCRIPTION OF THE INVENTION

The instant invention relates to the discovery that inhibiting tryptophan catabolism, and more particularly the inhibition of the activity of an IDO enzyme (e.g., IDO1 and/or IDO2), treats inflammatory diseases. Experimental evidence from a mouse model for rheumatoid arthritis is provided hereinbelow which demonstrates that pharmacological treatment with compounds that inhibit IDO1 and/or IDO2 can delay the onset and reduce the severity of symptoms associated with inflammatory disease progression. This is an unexpected finding as the current state of the art regarding IDO function in immune regulation would predict that inhibition of IDO enzymatic activity would exacerbate, not ameliorate, symptoms associated with an inflammatory disease such as arthritis. Without being bound to a particular theory, the data provided herein suggest that the primary immunomodulatory role of IDO in vivo is not to elicit T cell tolerance but rather involves shaping an immune response profile which supports the chronic pathological inflammation associated with a variety of disease states including, but not limited to, arthritis and cancer. This is in contrast to the current state of the art which has raised concerns that the treatment with IDO1 and/or IDO2 inhibitors would elicit or exacerbate autoimmune disorders or other inflammatory diseases (Munn et al. (2007) J. Clin. Invest., 117:1147-54; Puccetti et al. (2007) Nat. Rev. Immunol., 7:817-23; Penberthy, W. T. (2007) Curr. Drug Metab., 8:245-66; Puccetti, P. (2007) Eur. J. Immunol., 37:876-9; King et al. (2007) Int. J. Biochem. Cell Biol., 39:2167-72).

The instant invention also demonstrates synergy through the use of at least one IDO inhibitor and at least one other anti-inflammatory agent and/or immunosuppressive agent in the treatment of an inflammatory disease and in the inhibition of the onset of an inflammatory disease.

While the instant invention describes the use of IDO inhibitors for the treatment of inflammatory disease, inhibitors which block enzymes other than IDO which catalyze the degradation of tryptophan can be used, either alone or in coordination with an IDO inhibitor. For example, tryptophan 2,3-dioxygenase, encoded by the Tdo2 gene, catalyzes the same reaction as IDO and causes the degradation of tryptophan. As such, the inhibition of tryptophan 2,3-dioxygenase can be used to treat inflammatory disease.

### I. Definitions

As used herein, an “inflammatory disease” refers to a disease caused by or resulting from or resulting in inflammation. The term “inflammatory disease” may also refer to a dysregulated inflammatory reaction that causes an exaggerated response by macrophages, granulocytes, and/or T-lymphocytes leading to abnormal tissue damage and cell death. An “inflammatory disease” can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's Syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroditis, Graves' disease, Goodpasture's disease, mixed connective tissue disease, sclerosing cholangitis, inflammatory bowel disease, Crohn's Disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, and vulvovaginitis, angitis, chronic bronchitis, osteomylitis, optic neuritis, temporal arteritis, transverse myelitis, necrotizing fascilitis, and necrotizing enterocolitis. In a particular embodiment, the inflammatory disease is selected from the group consisting of arthritis, atherosclerosis, arteriosclerosis, ischemic injury, giant cell arteritis, inflammatory bowel disease, allergy, asthma, diabetes, lupus, multiple sclerosis, cystic fibrosis, hepatitis and psoriasis. In yet another embodiment, the inflammatory disease is selected from the group consisting of lupus, arthritis, and atherosclerosis.

The term “IDO inhibitor” refers to an agent capable of inhibiting the activity (e.g., the oxidoreductase activity) of indoleamine 2,3-dioxygenase (IDO1 and/or IDO2). Examples of IDO inhibitors are provided in PCT/US2008/57032, PCT/US2006/42137, PCT/US2004/005155, PCT/US2004/005154, U.S. patent applications Ser. Nos. 11/589,024, 10/551,151, and 10/550,444, and U.S. Provisional Patent Application 61/047,579 (these patent applications are incorporated by reference herein). In a particular embodiment, the IDO inhibitor is selected from the group consisting of 1-methyl-D-tryptophan, 1-methyl-L-tryptophan, 1-methyl-D/L-tryptophan, phenyl-thiohydantoin-trp (3-(N-phenyl-thiohydantoin)-indole), methyl-thiohydantoin-trp (3-(N-methyl-thiohydantoin)-indole), propenyl-thiohydantoin-trp (3-(N-allyl-thiohydantoin)-indole), 4-(butylamino)-3-hydroxy-2,2-dimethyl-3,4-dihydro-2H-benzo[g]chromene-5,10-dione (and hydroquinone derivative), 6-hydroxy-2,2-dimethyl-2H-benzo[g]chromene-5,10-dione (and hydroquinone derivative), napthalen-2-ylmethyl 2-(1H-indol-2-yl)ethylcarbamodithioate, pyridin-2-ylmethyl 2-(1H-indol-2-yl)ethylcarbamodithioate, 3-(1H-imidazol-4-yl)benzenethiol, 4-(1H-imidazol-4-yl)benzenethiol, and 2-(1H-imidazol-4-yl)phenol. In another embodiment, the IDO inhibitor is 1-methyl-D-tryptophan or a racemic mix comprising the same. In still another embodiment, the IDO inhibitor is 1-methyl-D-tryptophan.

As used herein, an “anti-inflammatory agent” refers to compounds for the treatment of an inflammatory disease or the symptoms associated therewith. Anti-inflammatory agents include, without limitation, non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin (see, e.g., Migita et al., Clin. Exp. Immunol. (1997) 108:199-203; Migita et al., Clin. Exp. Immunol. (1996) 104:86-91; Foroncewicz et al., Transpl. Int. (2005) 18:366-368), high density lipoproteins (HDL) and HDL-cholesterol elevating compounds (see, e.g., Birjmohun et al. (2007) Arterioscler. Thromb. Vasc. Biol., 27:1153-1158; Nieland et al. (2007) J. Lipid Res., 48:1832-1845; Bloedon et al. (2008) J. Lipid Res., Samaha et al. (2006) Arterioscler. Thromb. Vasc. Biol., 26:1413-1414, which discloses the use of rosiglitazone as an anti-inflammatory, Duffy et al. (2005) Curr. Opin. Cardiol., 20:301-306), rho-kinase inhibitors (see, e.g., Hu, E. (2006) Rec. Patents Cardiovasc. Drug Discov., 1:249-263), anti-malarial agents (e.g., hydroxychloroquine and chloroquine), acetaminophen, glucocorticoids, steroids, beta-agonists, anticholinergic agents, methyl xanthines, gold injections (e.g., sodium aurothiomalate), sulphasalazine, penicillamine, anti-angiogenic agents, dapsone, psoralens, anti-viral agents, statins (see, e.g., Paraskevas et al. (2007) Curr. Pharm. Des., 13:3622-36; Paraskevas, K. I. (2008) Clin. Rheumatol. 27:281-287), and antibiotics (e.g., tetracyclines). In a particular embodiment, the anti-inflammatory agent is an NSAID. In another embodiment the anti-inflammatory agent is methotrexate.

The terms “immunosuppressant” and “immunosuppressive agent”, as used herein, include compounds or compositions which suppress immune responses. Exemplary immunosuppressants include, without limitation, macrolides (e.g., pimecrolimus, tacrolimus (FK506), and sirolimus), cyclosporins (e.g., cyclosporin A), mycophenolate, azathioprine, dactinomycin, bucillamine, penicillamine, leflunomide, mercaptopurine, FK506, glucocorticoids, corticosteroids, purine analogs (e.g., azathioprine), pyrimidine analogs (e.g., cytosine arabinoside), mizoribine, alkylating agents (e.g., nitrogen mustard, phenylalanine mustard, buslfan, and cyclophosphamide), folic acid antagonsists (e.g., aminopterin and methotrexate), antibiotics (e.g., rapamycin, actinomycin D, mitomycin C, puramycin, and chloramphenicol), human IgG, antilymphocyte globulin (ALG), and antibodies (e.g., anti-CD3 (OKT3), anti-CD4 (OKT4), anti-CD5, anti-CD7, anti-IL-2 receptor (e.g., daclizumab and basiliximab), anti-alpha/beta TCR, anti-ICAM-1, anti-CD20 (e.g., rituximab), muromonab-CD3, anti-IL-12, alemtuzumab and antibodies to immunotoxins). In a particular embodiment, the immunosuppressive agent is an antibody (e.g., monoclonal) against CD20.

A “therapeutically effective amount” of a compound or a pharmaceutical composition refers to an amount effective to prevent, inhibit, treat, or lessen the symptoms of a particular disorder or disease. The treatment of an inflammatory disorder herein may refer to curing, relieving, and/or preventing the inflammatory disorder, the symptom of it, or the predisposition towards it. “Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, excipient, auxilliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin.

### II. Therapies and Compositions for the Treatment of Inflammatory Diseases

The present invention encompasses compositions comprising at least one IDO inhibitor and at least one pharmaceutically acceptable carrier. The composition may further comprise at least one other anti-inflammatory agent and/or at least one immunosuppressive agent. Alternatively, the at least one other anti-inflammatory agent and/or at least one immunosuppressive agent may be contained within a separate composition(s) with at least one pharmaceutically acceptable carrier. The composition(s) comprising at least one IDO inhibitor and the composition(s) comprising at least one other anti-inflammatory agent and/or at least one immunosuppressive agent may be contained within a kit. Such composition(s) may be administered, in a therapeutically effective amount, to a patient in need thereof for the treatment of an inflammatory disease. In a particular embodiment, the patient is monitored at least once for the inflammatory disease after administration of the compositions of the instant invention to monitor the treatment of the inflammatory disease (e.g., in the case of rheumatoid arthritis, joint (e.g., hand joint) pain and/or stiffness; presence of rheumatoid nodules; and/or presence of rheumatoid factor or rheumatoid factor antibodies in the blood).

The compositions of the present invention can be administered by any suitable route, for example, by injection (e.g., for local or systemic administration), oral, pulmonary, nasal or other modes of administration. In general, the pharmaceutically acceptable carrier of the composition is selected from the group of diluents, preservatives, solubilizers, emulsifiers, adjuvants and/or carriers. The compositions can include diluents of various buffer content (e.g., Tris-HCl, acetate, phosphate), pH and ionic strength; and additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435-1712 which are herein incorporated by reference. The pharmaceutical composition of the present invention can be prepared, for example, in liquid form, or can be in dried powder form (e.g., lyophilized).

In yet another embodiment, the pharmaceutical compositions of the present invention can be delivered in a controlled release system, such as using an intravenous infusion, an implantable osmotic pump, a transdermal patch, liposomes, or other modes of administration. In a particular embodiment, a pump may be used (see Langer, supra; Sefton, CRC Crit. Ref. Biomed. Eng. (1987) 14:201; Buchwald et al., Surgery (1980) 88:507; Saudek et al., N. Engl. J. Med. (1989) 321:574). In another embodiment, polymeric materials may be employed (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. (1983) 23:61; see also Levy et al., Science (1985) 228:190; During et al., Ann. Neurol. (1989) 25:351; Howard et al., J. Neurosurg. (1989) 71:105). In yet another embodiment, a controlled release system can be placed in proximity of the target tissues of the animal, thus requiring only a fraction of the systemic dose (see, e.g., Goodson, in Medical Applications of Controlled Release, supra, (1984) vol. 2, pp. 115-138). In particular, a controlled release device can be introduced into an animal in proximity to the site of inappropriate inflammation. Other controlled release systems are discussed in the review by Langer (Science (1990) 249:1527-1533).

The following examples are provided to illustrate various embodiments of the present invention. These examples are not intended to limit the invention in any way.

### EXAMPLE 1

K/BxN TCR transgenic mice express a TCR reactive to a self-peptide derived from the glucose-6-phosphate isomerase (GPI), presented by the MHC class II molecule A^{g7} (Korganow et al. (1999) Immunity, 10:451-461; Kouskoff et al. (1996) Cell, 87:811-822; Matsumoto et al. (1999) Science, 286:1732-1735). K/BxN mice spontaneously develop a very aggressive form of arthritis at 3 to 4 weeks of age. The arthritis of the K/BxN mice mimics arthritis in humans in that it is chronic, progressive, symmetrical, and exhibits the same histological features of human arthritis. The arthritis experienced by K/BxN mice is joint specific and allows for the scoring of the arthritis by caliper measurement of ankle thickness (Korganow et al. (1999) Immunity, 10:451-461; Ji et al. (2001) J. Exp. Med., 194:321-330).

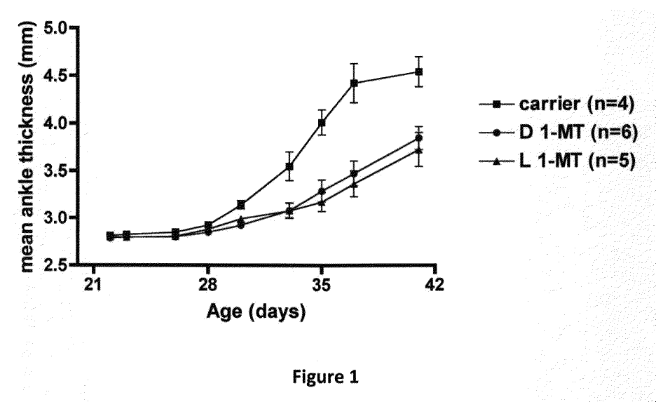

Starting at weaning (21 days of age) K/BxN mice were administered 400 mg/kg of 1-methyl-D-tryptophan, 400 mg/kg of 1-methyl-L-tryptophan, or carrier alone b.i.d. (twice daily) by p.o. (oral) gavage. As seen in FIG. 1, the administration of 1-methyl-D-tryptophan or 1-methyl-L-tryptophan delayed the onset of arthritis and greatly reduced the degree of arthritis experienced by the mice.

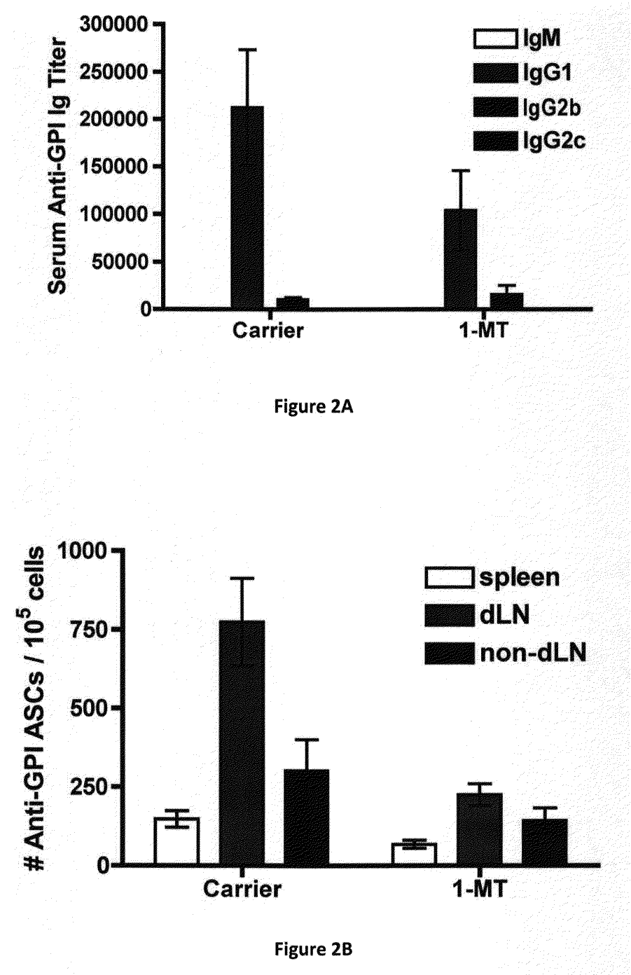

K/BxN mice produce arthritogenic Abs directed against GPI, which develop at high titers because of the preferential help that B cells expressing GPI-specific immunoglobulins receive from GPI-reactive T cells displaying the transgene-encoded TCR. As seen in FIGS. 2A and 2B, the serum of K/BxN mice administered with 1-MT (either D, L, or the racemic mix) had reduced levels of serum anti-GPI Ig compared to K/BxN mice administered with carrier alone and reduced numbers of anti-GPI antibody secreting cells in spleen, draining lymph nodes (dLN), and non-draining lymph nodes (non-dLN).

### EXAMPLE 2

Mice were given 400 mg/kg/dose (100 μl total volume) of D/L-1MT or D-1MT diluted in METHOCEL™/Tween® (0.5% Tween 80, 0.5% methylcellulose (v/v in water) twice daily by oral gavage (p.o.) using a curved feeding needle as described (Muller et al. (2005) Nat. Med., 11:312-319). 1MT was administered on a b.i.d. schedule, once in the morning and once in the evening. Methotrexate (MTX) was diluted in 0.5% carboxymethyl cellulose and administered by oral gavage at a range of doses 1-25 mg/kg/dose (0.1 cc/20 g mouse) using a curved feeding needle. The mice were given one dose per week as described (Asanuma et al. (2002) Eur. J. Pharmacol., 435:253-258). Control mice were given an equal volume of carrier alone (METHOCEL™/Tween®).

For the arthritis prevention experiments, MTX and/or 1MT treatment was started on the day of weaning (age 21 days). For the treatment of established arthritis, MTX and/or 1MT was administered after the mice had measurable arthritis.

The two rear ankles of MTX, 1MT, MTX+1 MT, or carrier-treated K/BxN mice were measured starting at weaning (3 wk of age) as described (Mandik-Nayak et al. (2002) Proc. Natl. Acad. Sci., 99:14368-14373). Measurement of ankle thickness was made above the footpad axially across the ankle joint using a Fowler Metric Pocket Thickness Gauge.

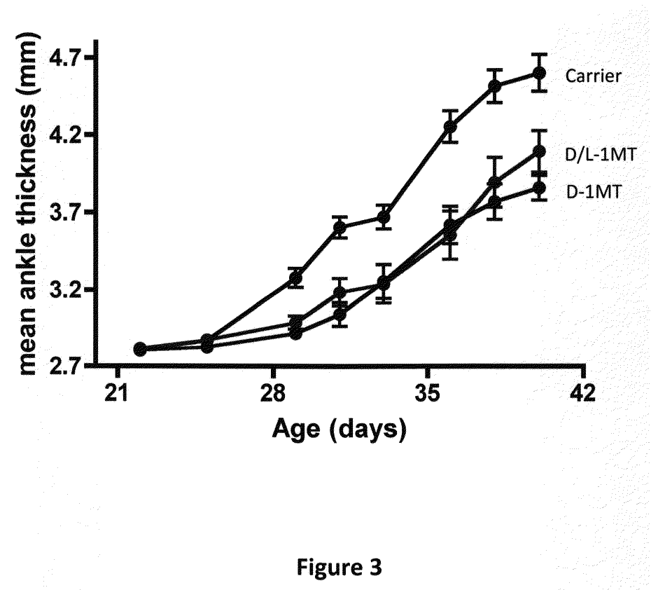

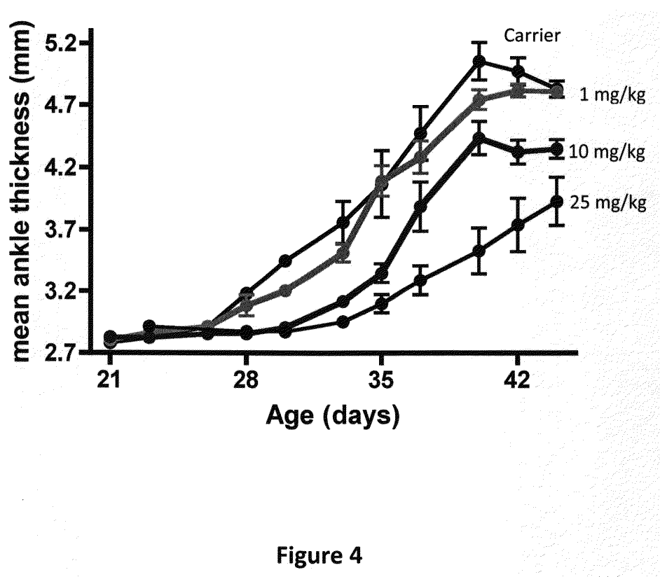

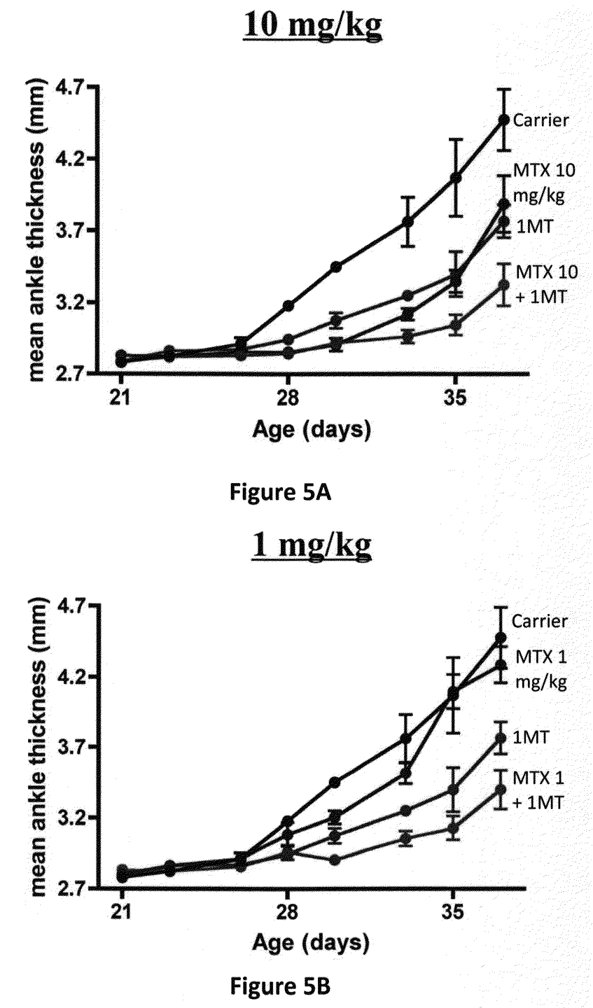

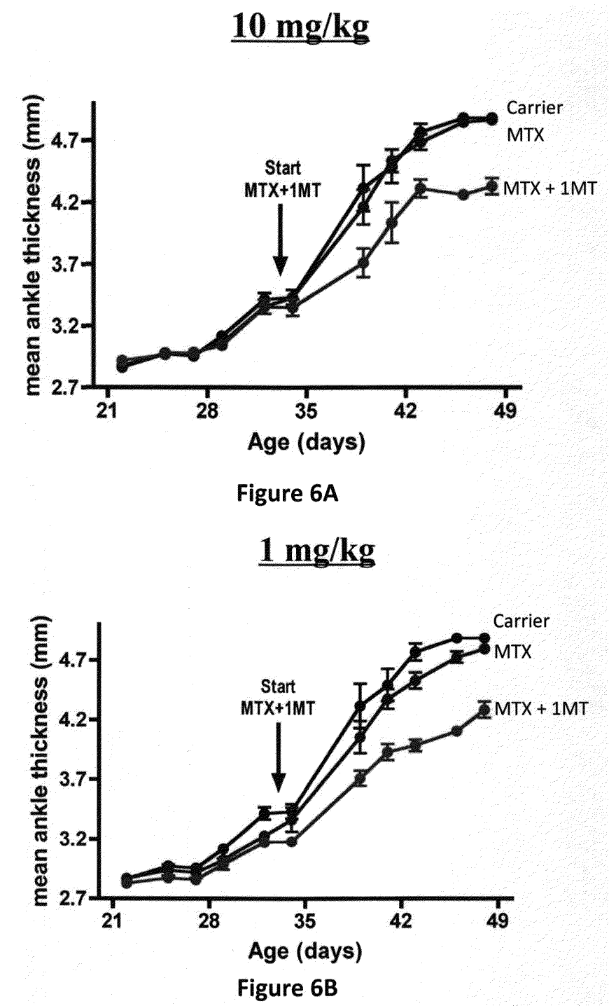

As seen in FIG. 3, the administration of 1-methyl-D-tryptophan or 1-methyl-D/L-tryptophan delayed the onset of arthritis and reduced the degree of arthritis experienced by the mice. FIG. 4 demonstrates that increasing amounts of MTX inhibited the onset of arthritis and reduced the degree of arthritis experienced by the mice. As seen in FIG. 5, the co-administration of 1MT and MTX significantly inhibited the onset of arthritis to a greater extent than either compound alone. As seen in FIG. 6, the co-administration of 1MT and MTX reduced the degree of arthritis experienced by the mice to a greater degree than the administration of the compounds individually.

### EXAMPLE 3

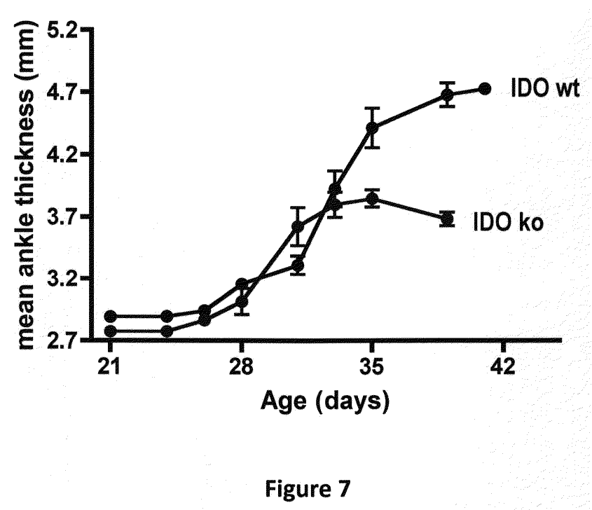

Arthritis was compared in IDO1 wild-type and IDO1 deficient KRN B6.g7 mice by measuring inflammation in the rear ankles. IDO1 deficient (IDO ko) arthritic mice were generated by breeding KRN IDO ko C57BL/6 mice with IDO ko mice expressing the I-Ag7 MHC Class II molecule to generate KRN/IDO ko B6.g7 mice. As seen in FIG. 7, IDO1 deficient mice have attenuated arthritis, thereby demonstrating that 1MT is targeting IDO in this model.

Several publications and patent documents are cited in the foregoing specification in order to more fully describe the state of the art to which this invention pertains. The disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for treating an inflammatory disease in a patient in need thereof, said method comprising the administration of at least one inhibitor of tryptophan catabolism.

2. The method of claim 1 comprising the administration of a composition comprising at least one inhibitor of at least one indoleamine 2,3-dioxygenase (IDO) and at least one pharmaceutically acceptable carrier.

3. The method of claim 2, wherein said at least one inhibitor inhibits indoleamine 2,3-dioxygenase-1 (IDO1).

4. The method of claim 2, wherein said at least one inhibitor inhibits indoleamine 2,3-dioxygenase-2 (IDO2).

5. The method of claim 2, wherein said at least one inhibitor inhibits both IDO1 and IDO2.

6. The method of claim 1, wherein said inflammatory disease is selected from the group consisting of arthritis, atherosclerosis, ischemic injury, giant cell arteritis, inflammatory bowel disease, allergy, diabetes, lupus, multiple sclerosis, cystic fibrosis, hepatitis and psoriasis.

7. The method of claim 6, wherein said inflammatory disease is rheumatoid arthritis.

8. The method of claim 1, wherein said method further comprises the administration of at least one additional anti-inflammatory agent.

9. The method of claim 1, wherein said method further comprises the administration of at least one immunosuppressive agent.

10. The method of claim 8, wherein said additional anti-inflammatory agent is methotrexate.

11. A composition comprising at least one inhibitor of at least one indoleamine 2,3-dioxygenase (IDO), at least one pharmaceutically acceptable carrier, and at least one agent selected from the group of additional anti-inflammatory agents and immunosuppressive agents.

12. The composition of claim 11, wherein said additional anti-inflammatory agent is methotrexate.
